(19) United States
(12) Patent Application Publication
(10) Pub. No.: US 2023/0110579 A1
(43) Pub. Date: Apr. 13, 2023

(54) CONTROL DEVICE, CONTROL METHOD, AND CONTROL PROGRAM

(71) Applicant: Murata Manufacturing Co., Ltd., Kyoto (JP)

(72) Inventor: Toru SHIMUTA, Kyoto (JP)

(21) Appl. No.: 18/064,997

(22) Filed: Dec. 13, 2022

Related U.S. Application Data
(63) Continuation of application No. PCT/JP2021/014327, filed on Apr. 2, 2021.

(30) Foreign Application Priority Data
Jun. 17, 2020 (JP) 2020-104509

(51) Int. Cl. G06F 16/25; G16H 40/60

(52) U.S. Cl. CPC G06F 16/258; G16H 40/60

(57) ABSTRACT

A system capable of efficiently analyzing data of a plurality of formats is provided. A control device includes a registration section that registers configuration information of custom formats that vary for different types of edge device, a reception section that receives data described in a custom format, a conversion section that identifies the configuration information of the custom format according to a device which is a transmission source of data received by the reception section and that converts the data into data of a common format which is redetermined, and a transmission section that transmits data converted by the conversion section to an analysis server including an analysis system that receives input of data of the common format.

### CROSS REFERENCE TO RELATED APPLICATION

This is a continuation of International Application No. PCT/JP2021/014327 filed on Apr. 2, 2021 which claims priority from Japanese Patent Application No. 2020-104509 filed on Jun. 17, 2020. The contents of these applications are incorporated herein by reference in their entireties.

### BACKGROUND ART

### Technical Field

The present disclosure relates to a control device, a control method, and a control program, each of which provides data for analysis.

In general, various physiological parameters such as medical waveform data obtained from patients by sensing are analyzed in a combined manner and used for determination of medical conditions and the like. In some cases, such analysis is automated using an analysis system that uses AI. In such cases, measurement data and the like, which are described in a predetermined common format, are input to the analysis system.

Devices such as sensors and the like each describes data using their own formats, and thus a conversion into the common format is necessary before analysis. With regard to techniques of the format conversion, for example, Patent Document 1 proposes a technique to perform a conversion of protocol format supported by each medical device during communications between medical devices.

Patent Document 1: Japanese Unexamined Patent Application Publication No. 2015-198941

### BRIEF SUMMARY

However, as described above, the formats of data output from devices are expected to be different in many ways. In order to input such a variety of data to the analysis system, a server for analysis needs to decode the formats of respective data and convert the decoded formats into the common format. Therefore, as the number of types of device increases, the processing load of the server increases.

The present disclosure provides a system capable of efficiently analyzing data of a plurality of formats.

A control device according to the present disclosure includes: a registration section that registers configuration information of a custom format, the custom format varying for different types of device; a reception section that receives data described in the custom format; a conversion section that identifies the configuration information of the custom format according to a device which is a transmission source of data received by the reception section and that converts the data into data of a common format which is predetermined; and a transmission section that transmits data converted by the conversion section to a server including an analysis system that receives input of data of the common format.

The conversion section may convert the data which is received into the data of the common format after adding unique information whose description is omitted in the custom format.

The registration section may obtain the configuration information of the custom format from a management server.

The registration section may obtain the configuration information, based on a device name obtained at a time of being connected with the device by communication.

The conversion section may add an ID of the control device to the data of the common format as information that identifies a user of the device.

The conversion section may add a time stamp to the data of the common format.

Data received by the reception section may include continuous data in time series.

The continuous data may be medical waveform data.

In a control method according to the present disclosure, a computer carries out: a registration step of registering configuration information of a custom format, the custom format varying for different types of device; a reception step of receiving data described in the custom format; a conversion step of identifying the configuration information of the custom format according to a device that is a transmission source of data received in the reception step and converting the data into a common format that is predetermined; and a transmission step of transmitting data converted in the conversion step to a server including an analysis system that receives input of data of the common format.

A control program according to the present disclosure is for causing a computer to function as the control device.

According to the present disclosure, it is possible to provide a system capable of performing data analysis efficiently.

### BRIEF DESCRIPTION OF THE DRAWINGS

FIG. 1 is a diagram illustrating an overall configuration of a data processing system according to an embodiment.

FIG. 2 is a diagram illustrating a functional configuration of a control device according to the embodiment.

FIG. 3 is a diagram illustrating an exemplary conversion of data format according to the embodiment.

### DETAILED DESCRIPTION

Hereinafter, an exemplary embodiment of the present disclosure will be described. FIG. 1 is a diagram illustrating an overall configuration of a data processing system 1 according to the present embodiment. The data processing system 1 includes a control device 10, edge devices 20, and an analysis server 30.

The control device 10 is connected with the edge devices 20 and the analysis server 30 by communication. The control device 10 is provided, for example, for each individual (patient), converts data transmitted from each of a plurality of the edge devices 20, and supplies the converted data to the analysis server 30. Note that the connection between the control device 10 and the edge device 20 may be a wired or wireless connection, and the communication protocol thereof is not limited. Further, the control device 10 may be connected to the analysis server 30 via a network, such as a LAN, the Internet, or the like.

The edge devices 20 include various communication devices, such as a sensor device that measures biological information of a patient, environment information, or the like, a camera device, an event switch, an information terminal, and the like. Data to be transmitted from the edge device 20 to the control device 10 include, for example, medical waveform data of, for example, electrocardiogram, brain waves, blood pressure, pulse waves, breathing, heart rate, oxygen saturation, body temperature, blood sugar level, blood flow, exhaled gas component, myopotential, magnetoencephalography, and the like, and further includes time-series continuous data of body surface temperature, amount of activity, posture (body position), perspiration, autonomic nerve function, depth of sleep, location (GPS), atmospheric pressure, atmospheric temperature, humidity, heat index (WBGT: wet-bulb globe temperature), illumination level, noise, and the like. Further, in addition to these continuous data, the edge device 20 may transmit image data, audio data, text data, and the like.

The analysis server 30 receives these data transmitted from the edge device 20 and converted by the control device 10. The analysis server 30 presents the received data to a user or performs data analysis by inputting the received data to the analysis system.

For example, in the determination of sleep apnea syndrome, a plurality of medical waveforms is referred. Specifically, in the case of obstructive sleep apnea syndrome (OSA), the airway is blocked but the breathing movement continues. On the other hand, in the case of central sleep apnea syndrome (CSA), the breathing movement is impeded. That is to say, if there is an up-and-down motion of chest after breathing (air flow through the mouth or nose) stops, it is OSA, and if not, it is CSA. In each case, however, the heart rate and the blood pressure increase, the oxygen saturation decreases, and the phenomenon of decreasing the depth of sleep occurs continuously. Accordingly, sleep apnea syndrome can be deduced by analyzing correlations among breathing, heart rate, blood pressure, oxygen saturation, depth of sleep, and the like.

Further, for example, as the analysis in which continuous data other than the medical waveforms are combined, there is the determination of heat stroke in which body temperature, heart rate, breathing, body surface temperature, amount of activity, posture (body position), perspiration, autonomic nerve function, location (GPS), atmospheric temperature, humidity, heat index (WBGT: wet-bulb globe temperature), illumination level, and the like are combined.

The analysis performed by a user or the analysis system may further use image information, text information, and the like. For example, the importance of event information (such as information on occurrence of seizure), which is generated irregularly by an event switch, increases when the event information is combined with waveform data.

Such a variety of data vary depending on the manufacturer and the type of device and are described in custom formats in many cases. Further, the continuous data have, for example, different appropriate sampling intervals as described below and vary depending on usages.
Electrocardiogram, blood pressure, pulse waves, blood flow, myopotential, magnetoencephalography, brain waves: Shorter than or equal to 10 msec
Heart rate: about 1 sec
Oxygen saturation, body temperature, blood sugar level, exhaled gas component, body surface temperature, amount of activity, posture (body position), perspiration, autonomic nerve function, depth of sleep, location (GPS), atmospheric pressure, atmospheric temperature, humidity, heat index (WBGT: wet-bulb globe temperature), illumination level, noise: Longer than or equal to 60 sec.

Because a data format for inputting to the analysis system is limited, the analysis server 30 needs to unify those custom formats into a common format. In the present embodiment, the control device 10 carries out this process of converting the custom formats into the common format, and this reduces the processing load of the analysis server 30 and facilitates the work of analyzing a plurality of data items in a combined manner.

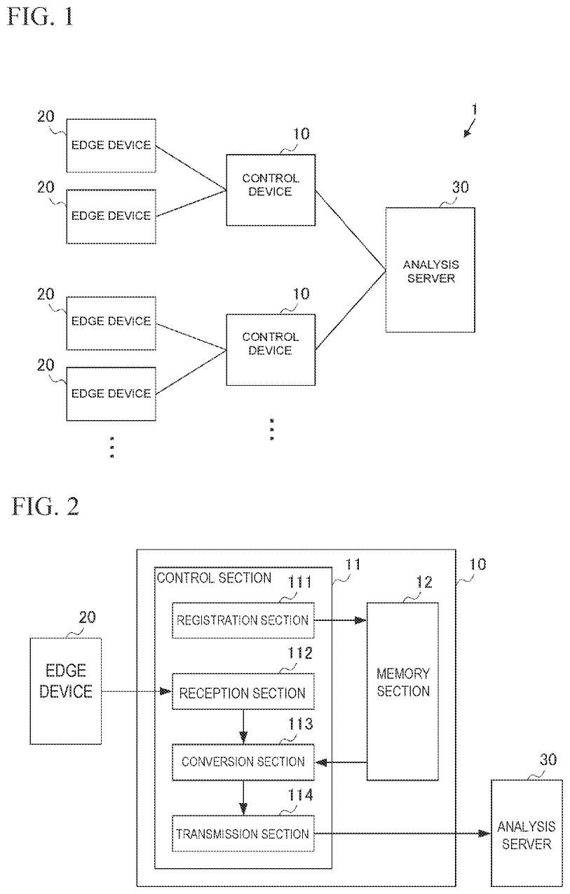

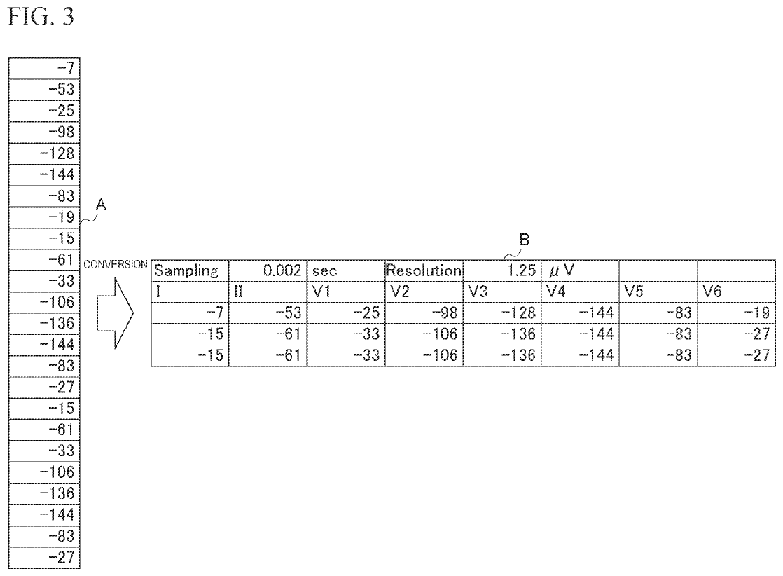

FIG. 2 is a diagram illustrating a functional configuration of the control device 10 according to the present embodiment. The control device 10 is an information processing device (computer), such as a smartphone, a tablet terminal, a personal computer, or the like, and includes, in addition to a control section 11 and a memory section 12, input/output devices for various data, a communication interface, and the like.

The control section 11 is a part that controls the entirety of the control device 10 and functions as a registration section 111, a reception section 112, a conversion section 113, or a transmission section 114 by appropriately reading and carrying out various programs stored in the memory section 12. The control section 11 may be a CPU (e.g., a processor).

The memory section 12 is storage space for various programs that cause a group of hardware components to function as the control device 10, various data, and the like. The memory section 12 may be a ROM, a RAM, a flash memory, a hard disk drive (HDD), or the like. Specifically, the memory section 12 stores a program (control program) that causes the control section 11 to implement respective functions of the present embodiment and further stores configuration information of data formats of respective edge devices 20 to be connected, configuration information of the common format, received data, and the like.

The registration section 111 registers the configuration information, such as data items, sequence, and the like of the custom format that varies for different types of the edge devices 20 based on input from a user and the like. Further, the registration section 111 may obtain the configuration information of the custom format from the analysis server 30 or another external server. At that time, the registration section 111 may obtain the configuration information by searching based on the device name, a unique ID of the edge device 20, or the like, which can be obtained at the time of being connected with the edge device 20 by communication.

The reception section 112 receives data described in a custom format that varies for different types of the edge devices 20. The data to be received by the reception section 112 include time-series continuous data such as medical waveform data and the like, as described above.

The conversion section 113 identifies the configuration information of the custom format according to the edge device 20 that is the transmission source of data received by the reception section 112, and converts the custom format of this received data into a common format that is predetermined. At that time, the conversion section 113 converts data of the custom format into data of the common format after adding unique information whose description is omitted in the custom format.

Further, the conversion section 113 adds an ID of the control device 10 to the data of the common format, which are data after the conversion, as information that identifies a user of the edge device 20. Moreover, the conversion section 113 adds a time stamp of the time of reception to the data of the common format, which are data after the conversion. Note that the common format may be a format according to a known standard such as MFER or the like.

The transmission section 114 (e.g., a transmitter) transmits the data of the common format, which are converted by the conversion section 113, to the analysis server 30.

FIG. 3 is a diagram illustrating an exemplary conversion of data format according to the present embodiment. In this example, time-series continuous data A are converted into data B to which various omitted data are added.

In the case where the edge device 20 to be connected to the control device 10 is pre-determined, the control device 10 registers a custom format of this edge device 20 in advance, interprets the continuous data A received from the edge device 20 based on the custom format, and converts the interpreted continuous data A into data B of the common format.

For example, unique information of the edge device 20, such as, for example, a sampling interval, a resolution power (resolution), channel information, and the like is omitted in the continuous data A of the custom format in order to reduce the volume of communication data. The control device 10 adds these omitted information items to the data B of the common format.

Specifically, for example, the following items are added based on the configuration information:
Sampling interval: A cycle of continuous data and 0.002 seconds in the illustrated example. The sampling interval may alternatively be specified by a sampling period [Hz].
Offset value: Actual observation value indicated by a digital value of 0. In the illustrated example, the offset value=0.
AD resolution power (resolution): For example, as the resolution power, 8 bits (256), 12 bits (4096), 24 bits (16777216), or the like is set. Alternatively, in the illustrated example, 1.25 μV is set as the minimum measurable unit corresponding to the resolution power. For example, when the offset value is 0, a digital value of “−128” is −128×1.25 μV=−160 μV.
Number of data (number of samples)
Data arrangement (number of channels, number of data blocks, sequence, and the like)

In the illustrated example, in the continuous data A that are data before the conversion, 8-channel time-series data observed at a sampling interval of 0.002 sec are sequentially described in a single row. In the data B that are data after the conversion, the sampling interval, the resolution power, and the identifiers of 8 channels (I, II, V1 to V6) are added, and 8-channel data are sequentially described in the respective rows.

Further, the data B that are data after the conversion are transmitted to the analysis server 30 after the ID of the control device 10 and the time stamp are added. Note that the sampling rates and time stamps of respective data received by the analysis server 30 vary, however, the analysis server 30 performs, if necessary, a process of interpolating data of low sampling rate to match data of high sampling rate or a similar process.

According to the present embodiment, the control device 10 registers the configuration information of custom formats that vary for different types of the edge devices 20 in advance, and upon receipt of data described in a custom format, identifies the configuration information according to the edge device 20 that is the transmission source of the data, converts the data into the common format, and then transmits the converted data to the analysis server 30. There are various types of edge devices 20 that measure biological information and the like, and in many cases, data formats vary depending on the manufacturer and the device type. Further, the sampling rate, the sampling timing, the data size, regular/irregular, and the like vary for different data, and thus it was difficult to uniformly process respective data at the time of analysis. In such a situation, even in the case where the edge device 20 to be connected changes, the control device 10 converts a variety of data into the common format in a similar manner and transmits the converted data to the analysis server 30. Accordingly, the processing load of the analysis server 30 decreases, and data analysis, particularly simultaneous analysis of a plurality of data items is easy to perform. As a result, the data processing system 1 capable of efficiently analyzing data of a plurality of formats is provided.

The control device 10 converts the received data into data of the common format after adding unique information whose description is omitted in the custom format. Accordingly, by providing the data after compensating information required in the analysis server 30, the control device 10 can reduce the processing load to be used for searching and adding the information in the analysis server 30.

The control device 10 obtains configuration information of custom formats from the analysis server 30 or another management server. This eliminates the need for registering information regarding the custom formats of data output from various edge devices 20 in each control device 10. Further, in the case the format is updated, there is no need for updating work of the registered information in each control device 10.

Based on the device name obtained at the time of being connected with the edge device 20 by communication, the control device 10 obtains information regarding the format of data from the analysis server 30 or another management server. This enables the control device 10 to automatically receive the information regarding the format that corresponds to the device name registered in the server, and thus it is possible to reduce the user's work of requesting the information from the server.

The control device 10 adds the ID of the control device 10 to the data of the common format as the information that identifies a user of the edge device 20. Suppose that the control device 10 performs analysis processes that fit individual users (for example, patients), personal information will be accumulated in the control device 10. This increases risks of information leakage. Further, at the time that the analysis server 30 analyzes data, identifying information is necessary to prevent mixing up of user data. However, an operation of registering a user ID using the control device 10 requires manpower, and input errors are likely to occur. Moreover, this necessitates storage of personal information, and thus, risks of information leakage increase. Therefore, by allowing the analysis server 30 to perform management while linking the ID of the control device 10 to the personal information of a user, from the ID of the control device added to data, it is possible to determine a user to whom the data belong. Accordingly, the mixing up of data is least likely to occur, and the personal information required for analysis is stored only in the analysis server 30. Thus, risks of leakage of the personal information from the control device 10 are low.

The control device 10 adds the time stamp to data of the common format. When respective data are transmitted or received, time lags are observed particularly in a network. Thus, if the time at which data reached the analysis server 30 were taken as the time of data acquisition, it is possible to have a long time lag depending on the communication environment in some cases. For that reason, the timing of adding time information can be as close as possible to the timing of generating data. However, in order to add time information (time stamp) at each edge device 20, it is necessary to install a real-time clock in the edge device 20. This may hinder the reduction of size, reduction of power consumption, and reduction of cost of the device in some cases. Accordingly, installing a real-time clock in the control device 10 can suppress the occurrence of a long time lag and further leads to the reduction of size, reduction of power consumption, and reduction of cost of the edge device 20.

The control device 10 converts time-series continuous data described in a custom format into data of the common format. This enables the data processing system 1 to analyze a variety of real-time data in a combined manner at low load. Particularly, by handing medical waveform data as continuous data, the data processing system 1 can simultaneously analyze a plurality of medical waveforms and efficiently perform an examination that requires a plurality of biological information items.

The embodiment of the present disclosure has been described. However, the present disclosure is not limited to the embodiment described above. Further, effects described in the embodiment described above are mere enumeration of most preferable effects produced by the present disclosure, and effects of the present disclosure are not limited to the ones described in the embodiment.

A control method achieved by the control device 10 is realized by software. In the case where the control method is realized by software, programs that constitute this software are installed in an information processing device (computer). Alternatively, these programs may be recorded on a removable media such as a CD-ROM and be distributed to a user, or may be distributed by downloading to a user's computer via a network. Moreover, these programs may alternatively be supplied to a user's computer as a Web service via a network without necessarily downloading.

### Reference Signs List

1 Data processing system

10 Control device

11 Control section

12 Memory section

20 Edge device

30 Analysis server

111 Registration section

112 Reception section

113 Conversion section

114 Transmission section

1. A control device comprising:
at least one processor configured to:
register configuration information of a custom format, the custom format varying for different types of devices,
receive data described in the custom format,
identify the configuration information of the custom format according to a transmission source device of the received data, and
convert the data into data of a predetermined common format; and
a transmitter configured to transmit the converted data to a server that includes an analysis system that receives input of data of the common format.

2. The control device according to claim 1, wherein the at least one processor is configured to convert the received data into the data of the common format after adding unique information whose description is omitted in the custom format.

3. The control device according to claim 1, wherein the at least one processor is further configured to obtain the configuration information of the custom format from a management server.

4. The control device according to claim 3, wherein the at least one processor is configured to obtain the configuration information based on a device name obtained at a time of connection with the transmission source device.

5. The control device according to claim 1, wherein the at least one processor is further configured to add an identifier (ID) of the control device to the data of the common format as information that identifies a user of the transmission source device.

6. The control device according to claim 1, wherein the at least one processor is further configured to add a time stamp to the data of the common format.

7. The control device according to claim 1, wherein the received data includes continuous data in time series.

8. The control device according to claim 7, wherein the continuous data is medical waveform data.

9. A control method carried out by a computer, the control method comprising:
registering configuration information of a custom format, the custom format varying for different types of devices;
receiving data described in the custom format;
identifying the configuration information of the custom format according to a transmission source device of the received data;
converting the data into a predetermined common format; and
transmitting the converted data to a server that includes an analysis system that receives input of data of the common format.

10. The control method according to claim 9, further comprising:
adding unique information whose description is omitted in the custom format before converting the received data into the data of the common format.

11. The control method according to claim 9, further comprising:
obtaining the configuration information of the custom format from a management server.

12. The control method according to claim 11, wherein the configuration information is obtained based on a device name obtained at a time of connection with the transmission source device.

13. The control method according to claim 9, further comprising:
adding an identifier (ID) of the control device to the data of the common format as information that identifies a user of the transmission source device.

14. The control method according to claim 9, further comprising:
adding a time stamp to the data of the common format.

15. The control method according to claim 9, wherein the received data includes continuous data in time series.

16. The control method according to claim 15, wherein the continuous data is medical waveform data.
